(19) United States
(12) Patent Application Publication
(10) Pub. No.: US 2018/0126100 A1
(43) Pub. Date: May 10, 2018

(54) HAND-ACTUATED AEROSOL GENERATOR AND ITS USE

(71) Applicants: James Zhou Liu, Kendall Park, NJ (US); Yuehua GU, Kendall Park, NJ (US)

(72) Inventors: James Zhou Liu, Kendall Park, NJ (US); Yuehua GU, Kendall Park, NJ (US)

(21) Appl. No.: 15/570,331

(22) PCT Filed: May 16, 2016
(86) PCT No.: PCT/US16/32705

§ 371 Date: Oct. 28, 2017

Related U.S. Application Data
(60) Provisional application No. 62/162,712, filed on May 16, 2015.

(51) Int. Cl. A61M 15/00; A61M 11/06; A61M 11/02

(52) U.S. Cl. CPC A61M 15/0086; A61M 11/06; A61M 11/02; A61M 15/0016; A61M 2205/075; A61M 15/0066; A61M 2202/04; A61M 2202/064; A61M 15/0021

(57) ABSTRACT

Disclosed herein are hand-actuated aerosol generators, having an adjustable two-compartment medicament container (20) that holds a medicament in a liquid or a fine dry powder form, and connects to a fluid passage; a cap of said container with a jet-sprayer capacity; a mouthpiece (6) through which a medicament released from said jet-sprayer (2) can be inhaled into the respiratory tract by a patient without leakage; a hand-actuated positive air pressure supplier connected to the cap of said medicament container, and an optional spacer that increases inhalation efficiency.

### CROSS-REFERENCE TO RELATED APPLICATIONS

The present application claims priority to and the benefit of U.S. Provisional Application No. 62/162,712, filed May 19, 2015. The present application is also related to U.S. Provisional Application 62/136,483, filed Mar. 21, 2015, an International Application No. PCT/US16/23058, filed Mar. 18, 2016, an International Application No PCT/US16/32705, filed on May 16, 2016, and U.S. application Ser. No. 15/560,319, filed Sep. 21, 2017. Each of the aforementioned applications is incorporated herein by reference in its entirety.

### TECHNICAL FIELD

The present application relates to inhalation devices, systems, process and methods for delivering aerosol into the lungs to treat a variety of diseases.

### BACKGROUND

The present invention provides a new medical device to deliver a liquid and/or a fine powder medicament painlessly, easily, accurately and directly to the respiratory tract through an inhalation action. Presently, the majority of all known dry powder inhalers (DPIs) are breath-actuated inhalers. The function of these DPIs is dependent on their internal flow rate and the user's inhalation capacity. In addition, there is a need to ensure that different patients receive substantially the same delivered dose from the same breath-actuated DPI. An alternative to the breath-actuated DPI, the metered dose inhaler (MDI) relies on the use of a chemical propellant to deliver to the lung a special formulation of a liquid medicament in an aerosol form. Many atomizers and nebulizers cannot be used to deliver the medicament both in liquid and in fine powder formulation with an accurate dose in a short time. There is a long felt need for a safe, effective and easy to use medical device for the administration of medicines in liquid and/or dry powder forms for the treatment of human diseases or disorders. The present invention is a new medical device for administering an exact dose of a drug which is in either a liquid or a dry powder formulation. Using this unique new device, numerous compositions can be delivered directly into the upper and/or lower respiratory tract. The newly invented medical device is a hand-actuated aerosol generator (HAAG). Other aspects of the current invention include numerous therapeutic kits for treating asthma, chronic obstructive pulmonary disease (COPD), diabetes, obesity and many other diseases by delivering a small amount of the traditional or modern medicine using a HAAG directly into the respiratory tract. A fine powder or fine mist delivered painlessly, easily, accurately and directly to the respiratory tract through an effortless non-breath-actuated inhalation has numerous advantages over oral intake or injection.

### DESCRIPTION OF RELATED ART

An effective and safe therapy relies on a functional drug and its delivery. For treating respiratory diseases, particularly, inhalation is a non-invasive approach for drug administration. Metered dose inhalers (MDIs) are commonly used but MDIs must use a chemical propellant to push medication out of the inhaler. MDIs require the user to press down the canister and inhale the medication in a well-coordinated manner; as a result, MDIs have a high percentage occurrence of patient misuse, which can lead to overdose or under-dose of medication. Another disadvantage of using MDIs is the added cost of the very expensive propellant.

Many dry powder inhalers (DPIs) have been developed and demonstrated many advantages over MDIs. DPIs in general are safer than the MDIs and cause fewer irritating side effects. The active ingredient(s) can be directly inhaled into the lungs with a DPI without using any preservatives or propellant. The state of art DPIs, however, also have many disadvantages, and hence limited their potential to become one of the most common devices for drug administration. A search for US patents with the phrase “dry powder inhaler” (DPI) yielded 3482 patents on Jan. 25, 2015. These DPIs belong to the “breath-actuated DPI,” or negative pressure DPI (NP-DPI). There is a critical need to reduce the flow rate dependence of breath-actuated DPIs, and, in particular, the flow rate dependence of the delivered dose of the medicament they deliver. In addition, there is a need to ensure that different patients receive substantially the same delivered dose from the same type of DPI.

Traditional Chinese medicine (TCM) has a history of several thousand years. Its origin could be traced back to remote antiquity. During its practice and evolution, TCM has been developed into a unique and integrated theoretical and practical system of medicine. Historically, there are roughly 13,000 medicines used in TCM and over 100,000 medicinal recipes recorded in the literature. Currently, TCM is an important part of medicine practiced in China and in many other countries. The most common route of administration of the TCM therapies is through oral intake. Although injection, patch, spray and other forms of administrations of TCM therapies have also been developed, they are less common. To further improve the effectiveness of TCM therapies, a more efficient delivery device is needed and it is provided in the current invention.

It is known that the oral intake of certain modern medicine, such as epinephrine, is less efficient comparing to direct delivery of the same drug, even in a much smaller amount, to the lower respiratory tract to treat allergy or respiratory diseases. The same is true for many TCM therapeutic formulae. Since efficient delivery of certain modern drugs to the lung is in practice by using breath-activated DPIs, efficient delivery of TCM formulae can also be carried out using the newly invented HAAG.

Many human diseases, such as asthma, chronic obstructive pulmonary diseases (COPD), diabetes, stroke, hypertension, depression or obesity, require long-term treatments. The ideal therapy should be easily delivered, have few or no side effect, and the effects can be experienced quickly. Currently, many drugs are available but their dosage-efficacy and side effects can be improved; it is desirable to use less chemicals, have faster action and less adverse effects. The hope is to use the newly invented HAAG to deliver many of the traditional and modern medicines in a small amount while to achieve the needed therapeutic effect.

Biologics represent a new category of drugs and have rapidly gained momentum in recent years. Antibodies and their derivatives, particularly human monoclonal antibodies, are a rapidly growing category of targeted therapeutic agents. In addition, small interfering RNA, cytokines, enzymes, and a variety of peptide drugs are being studied. Rapid discoveries of new drug targets, more effective engineering processes, and knowledge on the fate of the biologics in the body resulted in an increased number of biologics being on the market or in the late phases of clinical testing. Although these drugs are manufactured using advanced technologies, most of these proteins and peptides are delivered via the injection route, hence suffering all limitations linked to invasive drug delivery. As most of the therapy targets for biologics are chronic diseases, the limitations of invasive delivery are even more pronounced. Protein therapeutics offer a highly specific and rather complex set of functions, enjoy limited interference with the normal biological processes and low immunogenicity, and have the potential to replace gene therapy, and, from an industrial point of view, provide faster clinical development and approval time as well as better patent protection.

There are about 200 therapeutic proteins on the market, of which about 10% have been rationally designed with respect to their pharmacokinetics. For example, over 20 monoclonal antibodies have been approved by the US Food and Drug Administration and the European Medicines Agency and are currently the fastest growing category of targeted therapeutic agents and are expected to retain the same attractiveness.

In parallel, the fact that an increasing number of protein-based biologics are coming off patents in the next few years led to an extensive search for new products or formulations that can be patented by the pharmaceutical industry. New technologies enabling the improved delivery of biologics, such as needle-free devices, nanoparticles, and smart nanomaterials, together with the introduction of the concept of personalized medicine, resulted in their rapid market growth. Advanced delivery strategies could improve targeted delivery of RNA drugs, enabling maximized drug potency while minimizing off-target toxicity and immunogenicity.

As in all drug therapies, an efficient and targeted delivery of biologics to the desired site of action is the ultimate goal. However, due to their unique features, biologics represent a specific challenge in formulation development. Most often, strategies used in the product development of small molecular weight drugs cannot be readily transferred into the product development of biologics. Modified/improved strategies need to be applied to tackle the specific challenges linked to protein and peptide drugs. In addition, specific challenges and opportunities in nonclinical safety testing of biologics need to be addressed and optimized.

Most of the biologic products currently on the market are designed for the parenteral route of administration. For example, monoclonal antibody drug products have been on the market for over 20 years and are still administered in an acute care setting through intravenous infusion. Some of these products are designed for self-injection by patients, mostly as subcutaneous injections. Many biologics would greatly benefit from administration via alternative routes. For example, limitations exhibited by human growth hormone may be overcome by alternative routes to currently applied subcutaneous injections which would not only increase the compliance and convenience to the patients but also assure the required dosing accuracy, frequently an issue with subcutaneous administration. Intravenous (IV) and/or intramuscular (IM) infusion or injection has several disadvantages.

In response to the high occurrence of discontinuation of IV or IM treatment due to noncompliance, several new delivery devices have been proposed such as prefilled syringes, manual injector pens, auto-injectors, and needle-free devices. Significant improvements were also made in the field of micro-nanomechanical device-based drug delivery. These devices have shown potential in developing carriers with controlled physicochemical properties. A simple, easy to use and carry device for non-invasive administration of a variety of medicaments is expected to significantly increase the therapy compliance. The potential benefits of the current invention is to maximize drug potency while minimize off-target toxicity and immunogenicity.

Currently, intravenous (IV) nutritional therapies involve administering nutrients directly into the bloodstream (intravenously). This delivery system is quick and powerful because it circumvents the potential loss of potency due to possible breakdown in the gastrointestinal tract and poor absorption of some orally administered nutrients. Poor nutrition, chronic stress, improper sleeping patterns and lack of physical activity all contribute to premature aging, loss of skin elasticity, weight gain, abdominal fat, anxiety/depression, digestive disorders, skin problems, fatigue and illness. A healthy diet—high in vitamins, minerals, enzymes, phytochemicals, antioxidants, essential fatty acids, fiber and amino acids—along with proper sleep, exercise and stress management techniques, are all necessary for slowing the aging process. However, IV nutritional therapies need to be administered by a medical professional therefore very costly. The current invention will provide a much easier method to replace IV nutrition, in part.

### BRIEF SUMMARY

The present invention provides an easy to use, safe and effective medical device to administer traditional and modern medicines for the treatment of human diseases or disorders. The new device broadens the usefulness of traditional and modern medicines. Herb medicines have been used for thousands of years in China and in other countries, and now after a special preparation these natural drugs can be administered by using a newly invented hand-actuated aerosol generator (HAAG), a non-breath-actuated inhalation device. Similarly, many modern medicines have the same need to be delivered with a more suitable device for improved efficacy while decreasing their adverse effects. A small amount of fine powder or liquid mist can be delivered painlessly, easily, accurately and directly to the respiratory tract through an easy inhalation; such an approach to medicine delivery has many advantages as compared to those based on orally swallowing a huge amount of the same, or drug injection due to its non-invasive nature. This invention will reduce not only the potential side effects caused by allowing a large amount of the foreign chemical or biological materials enter into the human body, but also the waste of natural or synthetic resources. Other aspects of the current invention include numerous new kits for treating asthma, diabetes, obesity, depression and many other diseases or disorders by delivering an appropriate amount of the unique therapeutic powder or mist with a new HAAG into the lower respiratory tract.

### BRIEF DESCRIPTION OF THE DRAWINGS

FIG. 1 depicts an overall design of a hand-actuated aerosol generator for the delivery of a liquid or powder formulation according to the present invention when it is used in an upright position.

FIG. 2 depicts an overall design of a hand-actuated aerosol generator for the delivery of a liquid or dry powder formulation according to the present invention when it is used in an opposite down position.

FIG. 3 depicts the essential parts of a hand-actuated aerosol generator for the delivery of a liquid or a powder formulation according to the present invention when it is used in an upright position.

FIG. 4 depicts the essential parts of a hand-actuated aerosol generator for the delivery of a liquid or a dry powder formulation according to the present invention when it is used in an opposite down position.

FIG. 5 depicts the essential parts of a jet-sprayer head of said hand-actuated aerosol generator for the delivery of a liquid or dry powder aerosol according to the present invention.

FIG. 6 depicts the essential structure inside of said jet-sprayer head of said hand-actuated aerosol generator for the delivery of a liquid or dry powder aerosol according to the present invention.

FIG. 7 depicts another preferred embodiment of said hand-actuated aerosol generator for the delivery of a liquid or dry powder aerosol according to the present invention.

FIG. 8 depicts a preferred embodiment of a mouthpiece of said hand-actuated aerosol generator for the delivery of a liquid or dry powder aerosol according to the present invention.

FIG. 9 depicts a preferred embodiment of a dose-maker of said hand-actuated aerosol generator for the delivery of a liquid or dry powder aerosol according to the present invention.

FIG. 10 depicts a preferred embodiment of a carrying case of said hand-actuated aerosol generator for the delivery of a liquid or dry powder aerosol according to the present invention.

FIG. 11 depicts a preferred embodiment of a positive pressure supplier of said hand-actuated aerosol generator for the delivery of a liquid or dry powder aerosol under the standardized pressure according to the present invention.

FIG. 12 depicts a preferred embodiment of a positive pressure air supplier of said hand-actuated aerosol generator for the delivery of a liquid or dry powder aerosol under the standardized pressure according to the present invention.

FIG. 13 depicts a preferred embodiment of a spacer of said hand-actuated aerosol generator for the delivery of a liquid or dry powder aerosol according to the present invention.

### DETAILED DESCRIPTION

By integrating a hand-actuated positive pressure air-supplier (or narrowly termed air-pump) and a mouthpiece, certain nebulizer or atomizer can be transformed into a new controllable quantitative aerosol generator for use in oral inhalation. The newly created aerosol generator is a non-breath-actuated inhalation device, and it is called as a hand-actuated aerosol generator.

FIG. 1 shows the flow direction of a fluid flowing inside a hand-actuated aerosol generator when the device is held and operated in an upright position. In this position, a fluid transferring tube inside of the bottle is needed. The fluid is driven up, through the tube, by the positive pressure supplied by a hand-actuated device, such as a rubber bulb, or a hand-actuated air-pump. The fluid containing a dissolved medicament flows from the bottle to the jet-sprayer to become an aerosol, which then flows into the mouthpiece for oral inhalation. This is a sealed system and no leakage can happen. Therefore, the medicament can be administered without being released into the open air.

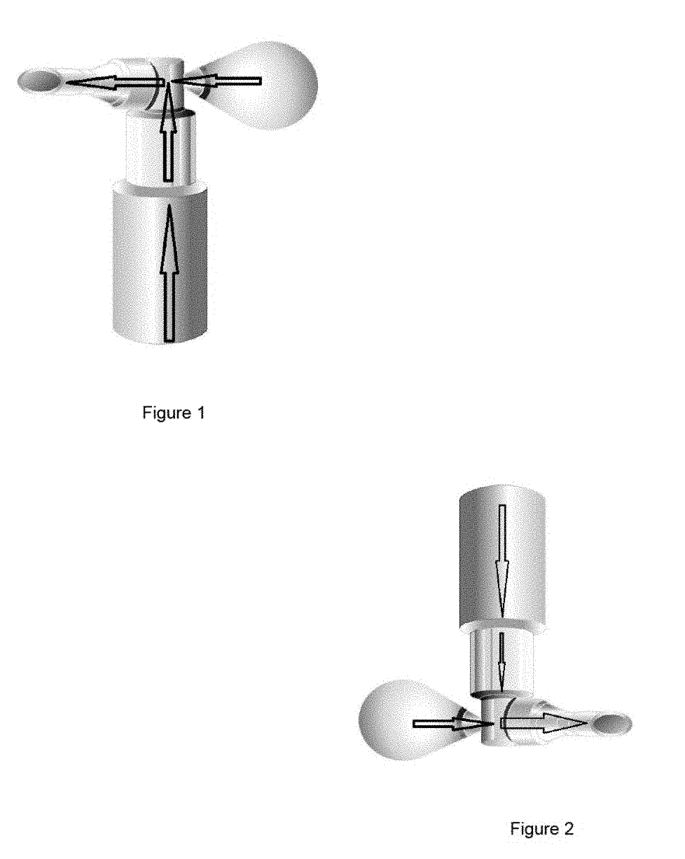

FIG. 2 shows the flow direction of a fluid flowing inside a hand-actuated aerosol generator when the device is held and operated in an upside-down position. In this position, no fluid transferring tube inside of the bottle is needed. The fluid is driven by the positive pressure supplied by a hand-actuated device, such as a rubber bulb, or a hand-actuated air-pump. The fluid containing a medicament, either a liquid or a dry powder, flows from the container to the jet-sprayer to become an aerosol, which then flows into the mouthpiece for oral inhalation. This is also a sealed system and no leakage can happen. Therefore, the medicament can be administered without being released into the open air.

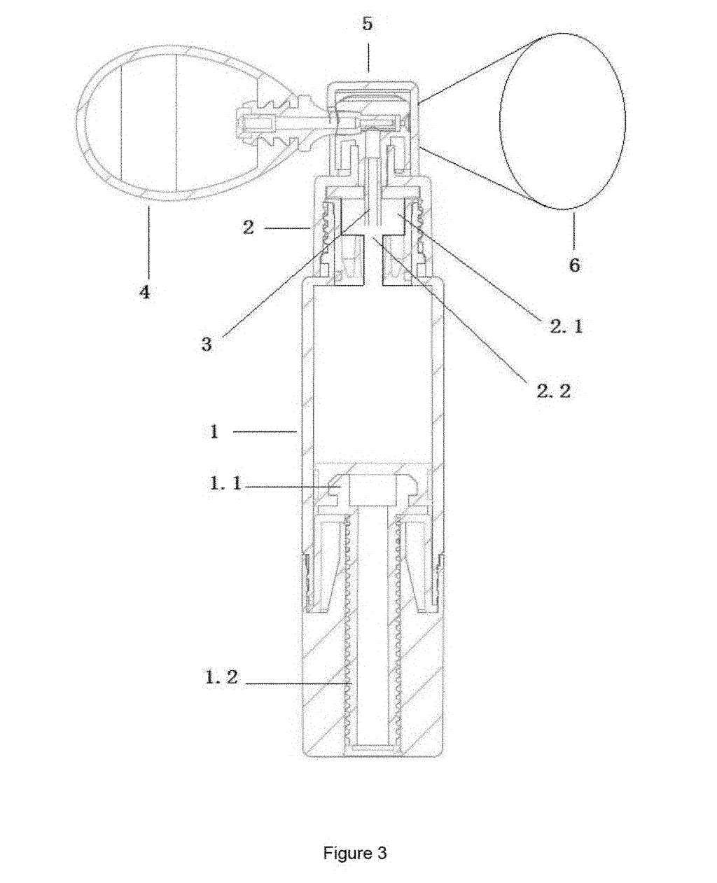

Shown in FIG. 3, a preferred embodiment of a hand-actuated aerosol generator comprises a number of essential parts: a) a medicament container 1 to hold either a liquid or dry powder drug. When the positive air supplier 4 is hand-actuated, the liquid or power held inside the dose-maker 2.1 is pushed to flow to a jet-sprayer head 2 to become an aerosol, which then flows towards the mouthpiece 6. A new dose is loaded inside of the dose-maker 2.1 through the one-way valve 2.2 from said container 1. The small tube 3 transfers the fluid or powder towards said dose-maker 2.1. The said mouthpiece 6 is clip-connected with the top 5 of said jet-sprayer 2 to be easily taken off for cleaning after each use. A patient can inhale the dosed-aerosolized medicament via said mouthpiece 6 without any leakage. In another preferred embodiment, the dosing volume 2.1 and the valve 2.2 can be designed to be a part of said medicament container 1. In that case, the dosing volume 2.1 and the valve 2.2 are placed at the neck-section of the medicament container. The volume of said space 2.1 can be varied and adjusted according to the weight or volume of a special drug. To fill the inner space of dose-maker 2.1, the user can turn one time the spiral-rod 1.2 inside of the medicament container 1 to force the top-surface 1.1 move upper-wards precisely in a defined height. This can fill the exact one dose to the Dose-Maker 2.1.

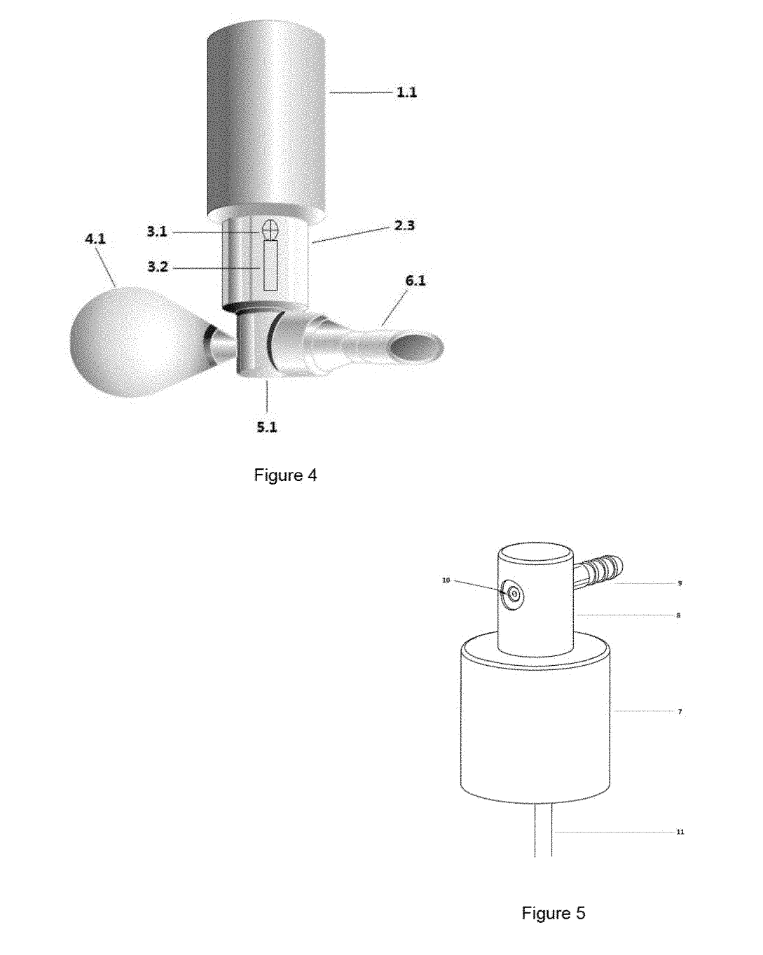

Shown in FIG. 4, another preferred embodiment of a hand-actuated aerosol generator, similar in design as shown in FIG. 3, comprises a number of essential parts: a) a medicament container 1.1 to hold either a liquid or dry powder drug. When the positive air supplier 4.1 is hand-actuated, the fluid or powder is pushed to flow from said container 1.1 to a dose-maker 3.2 via a one-way valve 3.1, then to the jet-sprayer head 2.3 to become an aerosol, before it flows towards the mouthpiece 6.1. The said mouthpiece 6.1 is clip-connected with the top 5.1 of said jet sprayer 2.3. A patient can inhale the dosed-aerosolized medicament via said mouthpiece 6.1 without any leakage. Inside said jet-sprayer, there is a defined space 3.2 to hold the medicament. Each dose of the medicament is determined by the pre-measured volume of said space 3.2. The volume of said space 3.2 can be varied and adjusted according to the weight or volume of a special drug. A valve 3.1 is placed there to allow the medicament to enter into the dose-maker space only, while preventing the medicament from flowing back to the container 1.1. In another preferred embodiment, the dose-maker 3.2 and the valve 3.1 can be designed to be an integral part of said medicament container. In that case, the dose-maker 3.2 and the valve 3.1 are placed at the neck-section of the medicament container.

FIG. 5 depicts various parts of a preferred embodiment of the head section of a jet-sprayer. Part 9 is the air-inlet through which the positive air pressure powers the jet-sprayer 8 and moves medicament towards the air-outlet 10 to become an aerosol, then enters into said mouthpiece as shown in FIGS. 3 and 4. Inside Part 7 there are several mechanical parts (not shown) to force a fluid to move towards said jet-sprayer 8. A tube 11 is needed to transfer the fluid-medicament under a negative pressure.

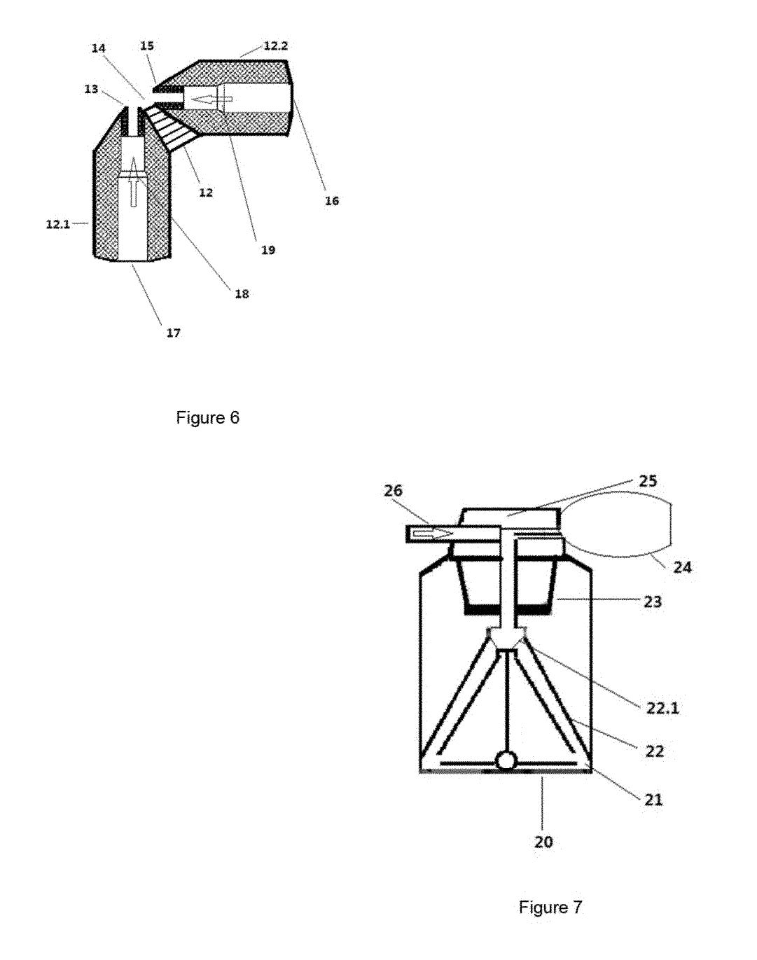

FIG. 6 depicts essential parts inside a jet-sprayer head of a hand-actuated aerosol generator. Parts 18 and 19 are valves to allow the fluid move only to one direction as shown. Space 14 is a small hollow space between the two sharp points 13 and 15. When the liquid or powder inside the space 17 moves to said space 14 under the sucking pressure generated by strong positive air-flow from air-inlet 16, the fluid is transformed into an aerosol in said space 14. The supporting structures 12, 12.1 and 12.2 secure the relative positions of all essential parts of 13, 14, 15, 16, 17, 18 and 19.

Shown in FIG. 7, another preferred embodiment of a hand-actuated aerosol generator comprises a number of essential parts. A medicament container 20 is used to hold either a liquid or a dry powder drug. A dose-maker 23 holds the medicament dose, which is determined by the pre-measured volume. The volume of said dose-maker 23 can be varied and adjusted according to a special drug. Also shown are the transferring tube 22, valves 22.1, the mouthpiece 24, the positive air supplier 26 and the jet-sprayer head 25. When the positive-air supplier 26 is hand-actuated, the fluid is sucked to flow from said medicament container 20 to a dose-maker 23 via tube 22, it then flows towards the jet-sprayer head 25 to become an aerosol, which enters into said mouthpiece 24. The said mouthpiece 24 is connected with the top 25 of said jet-sprayer. A patient can inhale the dosed-aerosolized medicament via said mouthpiece 24 without any leakage. Inside said jet-sprayer 25, there is a defined space 23 to hold the medicament in a pre-determined dosage. The holding volume of the dose-maker 23 can be varied and adjusted depending on the need of the actual pharmaceutical manufacturing practice and clinical objective. A valve 22.1 is placed to the dose-maker 23 to allow the dosed medicament enter into the dose-maker only, while preventing the medicament from flowing back to the container 20. Part 21 is a rotatable apparatus to move the liquid or powder towards the opening of said sucking tubes 22.

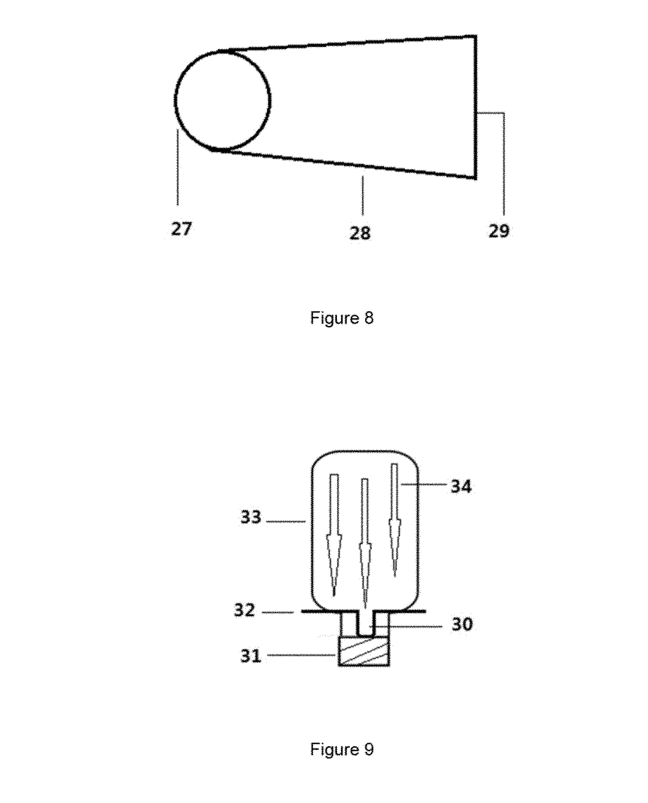

FIG. 8 depicts a preferred embodiment of a mouthpiece of a hand-actuated aerosol generator. Part 27 is connected with the jet-sprayer head tightly in a key-lock style. Part 28 forms the surrounding wall of the mouthpiece. The shape of said surrounding wall and the open-end 29 can be designed in any use-friendly way. Part 29 is the open-end through which a patient can orally inhale the dosed-aerosolized medicament. By having this critical mouthpiece, said aerosols can be retained in a specially sealed space, not freely flow around in the air after being released from said jet-sprayer. This ensures that an accurate dose, in the liquid or dry powder formulation, is completely administered into the body of a patient.

FIG. 9 depicts a preferred embodiment of a dose-maker as a part of the medicament container of a hand-actuated aerosol generator. Part 31 tightly connects with the jet-sprayer head. Part 30 is a defined hollow space inside of the container 33. The volume of dose-maker 30 can be varied and adjusted by using a hand or electricity, depending on the actual dosage of the pharmaceutical product and manufacturing process. Part 32 is an open-close switch to allow the fluid to enter the dose-maker 30 and prevent its backflow. Said switch 32 can be a simple shutter operated by a patient, or electronically powered by a battery. A spring structure may be a part of the switch to keep the dose-maker in the close or open position. It opens or closes when a patient to place this switch to an open or close position to load the dose. Upon the user places the switch at the close position, the dose is fulfilled and fixed. This close-open function can be realized by other common means, such as moving a central-hollow-ball to different alignment positions. It opens when a patient is to move, push or pull the switch, depending on the different designs. The indicator of the close-open position can be labeled nearby the physical switch to show which is a dose or an open position. Part 33 is the container of a medicament. Arrows 34 shows that under gravity, a liquid or a dry powder automatically fall down into the dose-maker 30 via the open-close switch 32. This dose maker is very unique as a part of the current invention. It can be used in the other medical devices to secure and deliver an accurate dose.

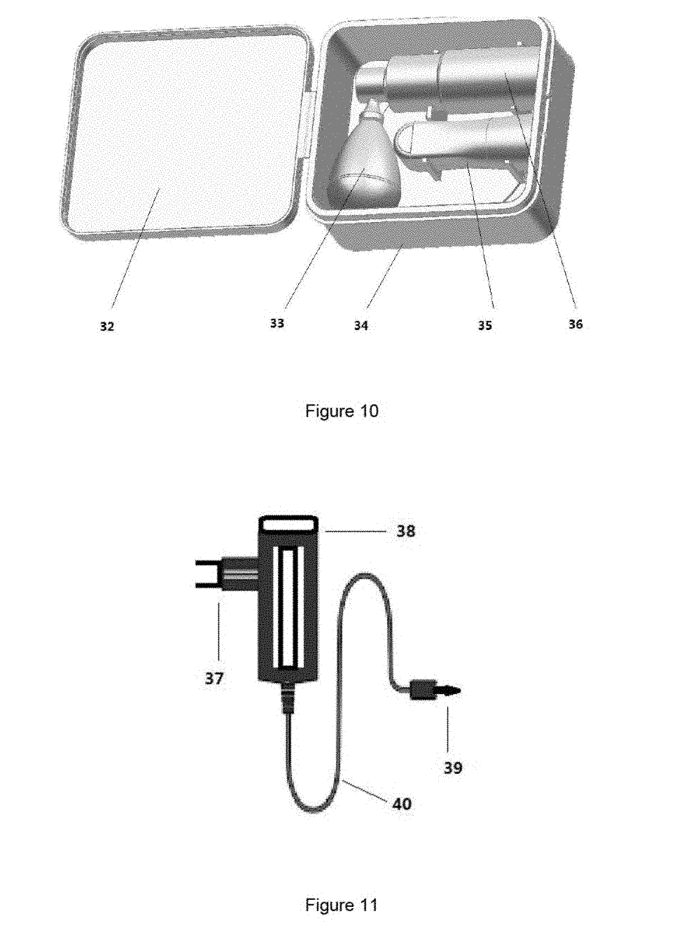

FIG. 10 depicts a preferred embodiment of a carrying case of a hand-actuated aerosol generator. Part 32 is the cover of the carrying case. Part 34 is the bottom section of the carrying case. Said bulb 33, said mouthpiece 35 and said Jet-sprayer set 36 are seated in the defined position and place inside the space secured by Parts 32 and 34 when the case is dosed.

FIG. 11 depicts a preferred embodiment of a positive pressure air pump which can be used to supply a positive pressure to any hand-actuated aerosol generator. The air-pump 38 and an airflow adaptor 39 can be made as a part of an integrated aerosol generator set, or can be made as a kit to be used to supply the positive air-pressure to any aerosol generator. Part 37 represents an electricity plug in or a battery or a group of batteries; Part 40 is an air tube between the pressurized air supplier and an airflow adaptor. A monitoring meter (not shown) of air-pressure, and an on/off switch (not shown) are parts of said air-pump 38. Using this set, a majority of currently existing aerosol generators can be converted into the air-pressure controlled aerosol generators to deliver a consistently accurate dose to a patient.

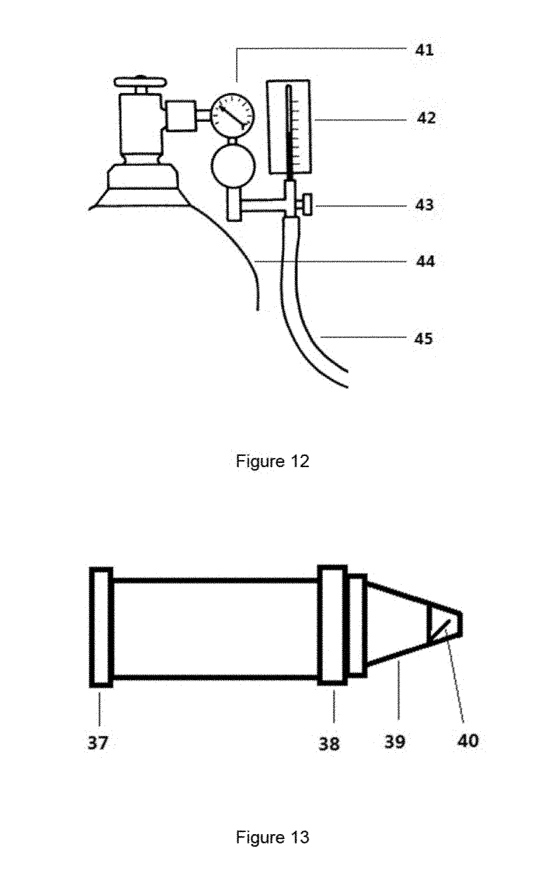

FIG. 12 depicts a preferred embodiment of a positive pressure air supplier which can be used to supply positive pressure to any hand-actuated aerosol generator. The pressurized air container 44 is the source of power. The airflow meter-adaptor 41 can be made as one set, or can be made as a kit to be used to supply positive air-pressure to any aerosol generator. Part 42 regulates the air flow rate such that a standard air-flow rate is maintained when it is in use. Part 43 is a hand-actuated switch. Part 45 is a tube to link the positive air to an aerosol generator. Using this set, a majority of currently existing aerosol generators can be converted into standard air-pressure controlled aerosol generators to deliver a consistently accurate dose to a patient.

FIG. 13 depicts a preferred embodiment of a spacer to be used with a hand-actuated aerosol generator. Part 48 is the head of the spacer to tightly connect with the jet sprayer head. Part 49 is a one-way valve to allow the aerosol to flow inside said spacer only. Parts 46 and 47 form a defined space to hold the aerosol. Part 46 can be linked with a mouthpiece for oral inhalation.

The hand-actuated aerosol generator is advantageous over existing nebulizers or atomizers in terms of delivery of an accurate dose per inhalation. It does not require a strong lung capacity for inhalation. This is particularly beneficial to old or very young patients, or a patient with COPD, who do not have a strong lung capacity. Using a hand-actuated aerosol generator enables the patient to inhale the liquid or powder drug without extra effort other than what is needed for normal inspiration.

Air-pumps suitable for use in a hand-actuated aerosol generator are widely available. These air pumps can be readily adapted to supply the positively pressurized air to an aerosol generator. Electric air-pumps are popular and well known, and can be easily controlled with a meter to indicate the supplied air flow rate. The present invention provides an adaptor to enable currently available electric air-pumps to become an important part of the new hand-actuated aerosol generator. The adaptor can connect the air-pump with a hand-actuated aerosol generator by a one-size-fit-all design and is preferably made with a special elastic and/or hard material. The tip of the adaptor is preferably soft and flexible which can be easily inserted into the air-inlet of any hand-actuated aerosol generator, and these DPIs.

Hand-held air pumps can also be adapted to supply air to a hand-actuated aerosol generator. The elasticity of the bulb and the volume of bulb will significantly impact the supplied air volume to the hand-actuated aerosol generator. These variations can be standardized after a few tests and the manufacturer can select the right size of the bulb and adjust its elasticity in order to supply the right amount of positive pressure under normal use.

Because the dosage is critically important for any therapy, the volume/size of the dose-maker of a hand-actuated aerosol generator is fixed and highly repeatable during any use. For any medicament, the accuracy of the specially needed doses can be realized by adjusting the volume of the dose-maker. The design of the simple adjustable dose-open switch for the dose-maker ensures that the device repeatedly supply a specially needed dose accurately for many therapies without a complicated electronic system. The cost for manufacturing is expected to be maintained at a very economical level.

It is known that many TCM therapies are available but these TCM formulas are rarely delivered using an inhalation device directly into a patient's lungs or lower respiratory tract. Now these TCM formulas can be delivered in a fine powder formulation by using a hand-actuated aerosol generator presented in the current invention. The present invention improves the efficacy of many TCM therapies which have a long-established successful use history. These TCM formulas are manufactured into a new liquid or dry powder form, packaged into a bottle or a vial, and then delivered by using a hand-actuated aerosol generator directly into the lower respiratory tract to treat respiratory diseases, such as acute bronchitis, acute respiratory distress syndrome (ARDS), asthma, acute or chronic bronchiolitis, bronchopulmonary dysplasia, chronic bronchitis, COPD, cystic fibrosis, emphysema, hantavirus pulmonary syndrome, hypersensitivity pneumonitis, influenza, lung cancer, pneumonia, primary pulmonary hypertension, pulmonary arterial hypertension, pulmonary fibrosis, pulmonary vascular disease, respiratory syncytial virus infection, severe acute respiratory syndrome, sleep apnea, or tuberculosis. The formulation, with minor adjustments, can also be used to treat upper respiratory tract diseases, such as common cold, sinusitis, allergic rhinitis, stridor, tonsillitis, epiglottitis, whooping cough (Pertussis), or croup.

Furthermore, the present invention enables the combination of two or more protective factors from TCM or modern medicines into a new formula where the new and combined formulation is better than using a single factor for managing asthma and for treating other respiratory diseases. One example is the use of a hand-actuated aerosol generator to directly deliver a dry powder containing both epinephrine and the extract of an herb in a very fine powder form. While epinephrine can dilate the lower respiratory tracts, the extract of Radix Scutellariae (Huang Qin) reduces inflammation of the lower respiratory tract. Other combinations with more than two protective factors can be delivered in the same way. The combination of several active ingredients from the herbal extracts can be safer and more effective than the drug made from a single chemical compound, particularly for those patients who are unable to achieve control of their asthma on an inhaled steroid alone. These combinational herbal formulae are formulated as a mixed fine powder or a liquid to be used for treating a human disease, such as asthma, by using a hand-actuated aerosol generator. These components in the original formulae can be extracted, spray dried to have the right particle sizes (2-10 micron) prior to being made into a fine powder, then inhaled via a hand-actuated aerosol generator into the lower respiratory tract to achieve a high therapeutic efficiency. Numerous herbs can be used for making the combinational formulae. Below are some of these herbs: Aloe vera (Lu Hui), Astragalus Root (Huang Qi), Codonopsis (Dang Shen), Belladonna alkaloids (Dian Qie Jian), Coix seed (Yi Yi Ren), Cordyceps militaris (Dong Chong Xia Cao), Cortex Mori Albae Radicis (Sang Bai Pi), egg shell, Flos Tussilaginis Farfarae (Kuan-Dong-Hua), Fructus Comi Officinalis (Shan Zhu Yu), Fructus Perilla Frutescens (Su Zi), Ginkgo (Yin Xing), Ginseng (Ren Shen), Glycyrrhizae (Gan Cao), Herba Ephedrae (Ma Huang), Jujube (Da Zao), Nonglutinous Rice (Geng Mi), Ophiopogonis (Mai Men Dong), Peppermint (Bo He), Pinelliae Preparatum (Zhi Ban Xia), Pseudo-ginseng (San Qi), Radix Dioscoreae Oppositae (Shan Yao), Radix Isatidis (Ban Lan Gen), Radix Rehmanniae Preparata (Shu Di Huang), Radix Scutellariae (Huang Qin), Rhizoma Alismatis Orientalis (Ze Xie), Safflower (Honghua), Salvia miltiorrhiza (Dan Shen), Scierotium Poriae Cocos (Fu Ling), Semen Pruni Armeniacae (Xing-Ren), Sophora flavescens ait (Ku Shen), Tripterygium wilfordii (Lei Gong Teng), or Tuber Pinellia (Ban Xia). It is noted that potentially suitable herbs are not limited to the above list, numerous other herb medicines are also candidates.

The formulae used in TCM can be made in the new dry fine powder form or a liquid form and delivered by using the hand actuated aerosol generator, as taught in the present invention. Thousands of such TCM formulae can be made in the dry powder form, suitable for delivery by using a hand-actuated aerosol generator. Below is an exemplary list of such TCMs which are suitable either alone or in combination with others for the new way of upper and lower respiratory delivery: An Zhong San (Calm the Middle Powder), Ba Wei Di Huang Wan (Eight-Ingredient Pill with Rehmannia), Ba Zhen Tang (Eight-Treasure Decoction), Bai He Gu Jin Wan (Lilium Teapills), Ban Xia Bai Zhu Tian Ma Tang (Pinellia, Atractylodes Macrocephala, and Gastrodia Decoction), Ban Xia Xie Xin Tang (Pinellia Decoction to Drain the Epigastrium), Bao Chan Wu You Fang (Preserve Pregnancy and Care-Free Decoction), Cang Er Zi (Upper Chamber Teapills), Chai Ge Jie Ji Tang (Bupleurum and Kudzu Decoction to Release the Muscle Layer), Chai Hu Gui Zhi Tang (Bupleurum and Cinnamon Twig Decoction), Chai Hu Qing Gan Tang (Bupleurum Decoction to Clear the Liver), Dang Gui Bu Xue Tang (Tangkuei Decoction to Tonify the Blood), Dang Gui Jing (Tang Kwei Gin), Dang Gui Liu Huang Tang (Tangkuei and Six-Yellow Decoction), Dang Gui Nian Tong Tang (Tangkuei Decoction to Lift the Pain), Dang Gui Shao Yao San (Tangkuei and Peony Powder), Dun Sou San (Long-Bout Cough Powder), Er Zhu Tang (Two-Atractylodes Decoction), Gan Lu Yin (Sweet Dew Decoction), Ge Gen Huang Qin Huang Lian Tang (Kudzu, Coptis, and Scutellaria Decoction), Gua Lou Zhi Shi Tang (Trichosanthes Fruit and Immature Bitter Orange Decoction), Gui Qi Jian Zhong Tang (Tangkuei and Astragalus Decoction to Construct the Middle), Gui Zhi Tang (Cinnamon Twig Teapills), Huai Hua San (Sophora Japonica Flower Powder), Huang Qi Jian Zhong Tang (Astragalus Decoction to Construct the Middle), Ling Gui Zhu Gan Tang (Poria, Cinnamon Twig, Atractylodes Macrocephala, and Licorice Decoction), Liu Wei Di Huang Wan (Six Flavor Teapills), Ma Huang Xing Ren Gan Cao Shi Gao Tang (Ephedra, Apricot Kemrnel, Licorice, and Gypsum Decoction), Mai Men Dong Tang (Ophiopogonis Decoction), Qiang Huo Sheng Shi Tang (Notopterygium Decoction to Overcome Dampness), Qing Bi Tang (Clear the Nose Decoction), Qing Fei Tang (Clear the Lung Decoction), Qing Hao Bie Jia Tang (Artemisia Annua and Soft-Shelled Turtle Shell Decoction), Qing Shang Fang Feng Tang (Clear the Upper and Guard the Wind Decoction), Qing Shu Yi Qi Tang (Clear Summer-Heat and Augment the Qi Decoction), Qing Xin Li Ge Tang (Clear the Epigastrium and Benefit the Diaphragm Decoction), Qing Xin Lian Zi Yin (Lotus Seed Decoction to Clear the Heart), Qing Zao Jiu Fei Tang (Eliminate Dryness and Rescue the Lung Decoction), Ren Shen Yang Ying Tang (Ginseng Decoction to Nourish the Nutritive Qi), San Huang Xie Xin Tang (Three-Yellow Decoction to Sedate the Epigastrium), San Zhong Kui Jian Tang (Disperse the Swelling and Break the Hardness Decoction), Shao Yao Tang (Peony Decoction), Shi Liu Wei Liu Qi Yin (Sixteen-Ingredient Decoction to Flow Qi), Shou Wu Zhi (Shou Wu Essence), Shu Jing Huo Xue Tang (Relax the Channels and Invigorate the Blood Decoction), Si Jun Zi Tang (Four Gentlemen Teapills), Si Ni San (Four Pillars Teapills), Si Wu Tang (Four Substances For Women), Tao He Cheng Qi Tang (Peach Pit Decoction to Order the Qi), Wei Ling Tang (Calm the Stomach and Poria Decoction), Wen Qing Yin (Warming and Clearing Decoction), Wu Zhu Yu Tang (Evodia Decoction), Xiang Sha Ping Wei San (Cyperus and Amomum Powder to Calm the Stomach), Xiao Chai Hu Tang (Minor Bupleurum Decoction), Xiao Cheng Qi Tang (Minor Order the Qi Decoction), Xie Huang San (Drain the Yellow Powder), Xin Yi Qing Fei Yin (Magnolia Decoction to Clear the Lung), Xue Fu Zhu Yu Tang (Stasis In The Mansion Of Blood Teapills), Yang Xin Tang (Nourish the Heart Decoction), Yi Gan San (Restrain the Liver Powder), Yin Chen Hao Tang (Artemisia Scoparia Decoction), Yin Qiao San (Honeysuckle and Forsythia Powder), Yu Ping Feng San (Jade Screen Teapills), Yu Nu Jian (Jade Woman Decoction), Zhe Chong Yin (Break the Conflict Decoction), Zhi Gan Cao (Honey-fried Liquorice Root), Zhi Sou San (Stop Coughing Powder), Zhu Ling Tang (Polyporus Decoction), Zhu Ye Shi Gao Tang (Bamboo Leaves and Gypsum Decoction), or Zi Yin Jiang Huo Tang (Nourish Yin and Descend the Fire Decoction). It is noted that potentially suitable TCM formulae are not limited to the above list—numerous other TCM medicaments are also candidates.

Many drugs used in modern medicine for specifically treating respiratory diseases have been delivered by using the best available device during the research and development phase. If the chemical drug was delivered by using a MDI or DPI, the same chemical drug can be potentially delivered using the newly invented hand-actuated aerosol generator to reduce the dosage variation due to differences in the patients' inhalation capacity. If these chemical drugs were delivered via injection, now these same chemicals can also be potentially made into a dry powder form or a liquid form and delivered using a hand-actuated aerosol generator to eliminate the pain, cost in administration and infection risk due to injection. If the drug was delivered though oral intake and then absorbed into blood circulation, that chemical can also potentially be re-formulated into a fine powder form or a liquid form then delivered by using a hand-actuated aerosol generator to improve the efficacy and to reduce their side effects.

A small group of drugs currently being administered using a breath-actuated DPI for the treatment of respiratory diseases can be delivered by using the newly invented hand-actuated aerosol generator to reduce the dosage variation. The drug ingredients are, but not limited to, albuterol, salmeterol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol, or (−)-4-amino-3,5-dichloro-alpha-[[[6-[2-(2-pyridinyl) ethoxy]hexyl]met-hyl]benzene-methanol. The drugs to be delivered in a powder form by using a hand-actuated aerosol generator can be in the form of salts, esters, etc., to thereby optimize the activity, efficacy and/or stability of the medicament.

A large group of the drugs currently used in modern medicine to treat a variety of diseases and/or disorders are delivered mainly through oral intake, injection, patch, spray and inhalation with other disadvantageous devices. Depending on the specific chemical and physical characteristics of the chemical or biological preparation, each drug has an optimal delivery route identified during the research and development stage of that drug, prior to the current invention of the hand-actuated aerosol generator. Broadly speaking, after overcoming these disadvantages of currently available non-positive pressure based dry powder inhalers, these drugs should be re-evaluated for their suitability for delivery using the new hand-actuated aerosol generator to improve efficacy and reduce side effects. Even for certain historically failed drugs due to their poor delivery route in the past, they may be revived if that drug can be appropriately delivered by using the new hand-actuated aerosol generator for developing any new drug. In other words, the new hand-actuated aerosol generator has a potential to become one of the most commonly used medical device to deliver a variety of therapies due to its numerous advantages and capabilities.

It is beneficial to the pharmaceutical industry and patients to consider using the new hand-actuated aerosol generator to deliver many medicines directly into the lungs for improving the therapeutic efficacy of these chemicals. Two basic requirements for selecting any chemical for delivery by using the hand-actuated aerosol generator are: (1) it is safe after being directly delivered into the lungs; (2) it is effective after entering into blood circulation though the lungs. Any drug meeting these two requirements can potentially be delivered by using the newly invented hand-actuated aerosol generator. Some examples are (but not limited to): acetaminophen and/or propoxyphene for arthritis; aclidinium, budesonide, formoterol, roflumilast, umeclidinium, vilanterol for COPD; adalimumab, leflunomide, tocilizumab for rheumatoid arthritis; albuterol, montelukast, methylprednisolone, mometasone/formoterol, or ipratropium for asthma; almotriptan, diclofenac, eletriptan for migraine alprazolam, amitriptyline, bupropion, fluoxetine, vilazodone for depression; alprostadil, sildenafil, tadalafil, vardenafil, for erectile dysfunction; alteplase, aspirin, dopidogrel for stroke; amifostine, chlorambucil, cyclophosphamide, fluorouracil, or vincristine for cancer; amitriptyline for depression; amlodipine besylate for hypertension; amoxicillin, ceftriaxone, ciprofloxacin for urinary tract infection; amphetamine sulfate for attention deficit hyperactivity disorder; aripiprazole, lurasidone, risperidone, ziprasidone for schizophrenia; atenolol for angina; atorvastatin calcium for osteoarthritis; atorvastatin, ezetimibe, pitavastatin, rosuvastatin, simvastatin for high cholesterol; azithromycin, levofloxacin, moxifloxacin for bronchitis; budesonide, sulfasalazine for inflammatory bowel disease; bupropion hydrochloride for depression; candesartan cilexetil for hypertension; carisoprodol for pain; ceftazidime for endocarditis; Celecoxib, Meloxicam, naproxen-esomeprazole for osteoarthritis; cetirizine, fluticasone furoate for hayfever; chlorpheniramine/acetaminophen, diphenhydramine got colds & Flu; clonazepam, lamotrigine, lurasidone for bipolar disorder; conjugated estrogens, zoledronic acid for osteoporosis; diethylpropion, lorcaserin, methamphetamine, phentermine for weight control; diphenhydramine for allergy; disopyramide, dofetilide for arrhythmia donepezil for Alzheimer's; duloxetine for depression; emtricitabine/rilpivirine/tenofovir for AIDS/HIV; enalapril, nebivolol, olmesartan, triamterene/hydrochlorothiazide for hypertension; epinephrine, epinephrine isomers, ephedrine for asthma and COPD; escitalopram for anxiety; esomeprazole for GERD (Heartburn); estazolam, flurazepam, olanzapine for insomnia; esterified estrogens/methyltestosterone for menopause disorder; divalproex sodium ethosuximide, levetiracetam, primidone for seizures; ezetimibe for high cholesterol; fluoxetine for depressive disorders; gabapentin for nerve pain; heparin sodium for blood clots in the veins, arteries, or lungs; hepatitis b vaccine for hepatitis B; hydrochlorothiazide for edema; hydrocodone bitartrate for severe pain; hydromorphone, oxycodone, tapentadol for pain; hyoscyamine, mirabegron for urinary incontinence; ibuprofen for inflammation and pain; immune globulin for hepatitis A; insulin, insulin glargine, insulin lispro, pramlintide for diabetes (Type 1); insulin detemir, liraglutide, repaglinide, empagliflozin for diabetes (Type 2); isotretinoin for acne; levocetirizine for allergies; levofloxacin for bacterial infections; levothyroxine, liothyronine, thyroid for hypothyroidism; lisdexamfetamine for ADHD; loratadine for allergy; lorazepam for anxiety disorders; morphine, oxycodone for moderate to severe pain; naproxen for arthritis pain or inflammation; nifedipine for hypertrophic cardiomyopathy; oseltamivir, zanamivir for influenza; paroxetine hydrochloride for depression; pegloticase for gout; penicillin for bacterial infection; phentermine for obesity; pregabalin for fibromyalgia; pregabalin for seizures; quetiapine fumarate for schizophrenia, ranolazine for angina; risperidone for schizophrenia and symptoms of bipolar disorder; sertraline hydrochloride for depression; sildenafil citrate for pulmonary arterial hypertension; tramadol hydrochloride for pain; valsartan for hypertension; vardenafil hydrochloride for impotence; venlafaxine hydrochloride for depression or zolpidem tartrate for sleep problems. These pharmaceutically active agents may be used in the form of salts, esters or solvates to thereby optimize the activity, efficacy and/or stability of the medicament. They can be easily administered in any combination as medically needed, since the potential in vitro interaction of different chemicals in the dry powder form will be reduced dramatically, or even eliminated.

Each ingredient has its required dosage for safety and effectiveness when it is delivered by using a hand-actuated aerosol generator. The dose of each of these current drugs in the marketplace or in the research and development phases was or will be established and these doses are served as references for deciding the dose in the hand-actuated aerosol generator formulation. In a broad range, the dosage of each of these pharmaceutical substances used in the formulation for the hand-actuated aerosol generator is expected to vary between 5 times higher to 100 times lower than its previously established dose using its established delivery route. Preferably, the same or several times less amount of the actual drug substance will be contained in a dose-maker of the hand-actuated aerosol generator as compared to the current dose of such a drug.

If the drug is currently made in an injection formulation, preferably, the amount of the actual drug substance contained in a dose-maker of the hand-actuated aerosol generator is the same as its corresponding injection formulation.

If the drug is currently made in an oral intake formulation, preferably, the same amount of the actual drug substance contained in a dose-maker of the hand-actuated aerosol generator is the same as its corresponding oral intake formulation. More preferably, at least 2-times less amount of the actual drug substance is contained in a dose-maker of the hand-actuated aerosol generator than that in its corresponding oral formulation. More preferably, at least 5-times less amount of the actual drug substance is needed in a dose of the hand-actuated aerosol generator formulation than that in its corresponding oral formulation.

If the drug is currently made in a patch formulation, preferably, the same amount of the actual drug substance contained in a dose-maker of the hand-actuated aerosol generator is the same as its corresponding patch formulation. Preferably, at least 2-times less amount of the actual drug substance is needed in a dose-maker of the hand-actuated aerosol generator than that in its corresponding patch formulation.

If the drug is currently made in an oral or nasal spray formulation, preferably, the same amount of the actual drug substance contained in a dose-maker of the hand-actuated aerosol generator is the same as its corresponding spray formulation. Preferably, at least 2-times less amount of the actual drug substance is needed in a dose-maker of the hand-actuated aerosol generator than that in its corresponding spray formulation.

If the drug is currently made in an aerosol formulation, preferably, the same amount of the actual drug substance contained in a dose-maker of the hand-actuated aerosol generator is the same as its corresponding aerosol formulation. Preferably, a two or more times less amount of the actual drug substance is needed in a dose-maker of the hand-actuated aerosol generator than that in its corresponding aerosol formulation.

In a specific embodiment, the dosage range of inhalable epinephrine in a formulation of a hand-actuated aerosol generator is between 1.1 times higher and 2.0 times lower than its actual amount in the inhalation formation, which are 0.10 mg and 0.25 mg per inhalation. Due to variations in the pharmaceutical industry and a patient's body size and age, the dry powder formation of the inhalable epinephrine for the formulation of a hand-actuated aerosol generator comprises approximately 0.01 mg to about 2.00 mg of epinephrine or its equivalent compounds; or alternatively about 0.05 mg to about 1.00 mg; or alternatively about 0.10 mg to about 0.50 mg; or alternatively about 0.15 mg to about 0.30 mg; or alternatively about 0.20 mg to about 0.25 mg; or alternatively about 0.22 mg to about 0.25 mg. The dry powder formulation may comprise about 95.00% to about 99.99% (w/w) of a carrier, such as inhalable lactose.

Another group of drugs are biologics, or so called big molecules. These biologics are not easy or suitable for oral intake as many factors in the digestive system could denature these big molecules. With the exception of certain big molecules which could cause toxicity if inhaled into the lungs, such as Abobotulinum Toxin Type A or Incobotulinumtoxin A, most biologics currently administered by injection or inhalation can be delivered by using the newly invented hand-actuated aerosol generator. In other words, hand-actuated aerosol generator delivery can be used to replace most of the injection form of the drugs to eliminate a number of drawbacks of the injectables—these include: difficulty in manufacturing the stable dose; difficulty in keeping a long shelf-life; difficulty in storage before use and during transportation (keep in refrigerator or a freezer, for example); pain and the risk of potential infection during and after injection/infusion, and the high cost and inconvenience caused by the need to perform the injection/infusion by a medical professional. These biologics would undergo a freeze-drying or dry spray process before being packed into a dry powder dosage for administering with a hand-actuated aerosol generator. A dry powder is more stable than an injection liquid. Disadvantages associated with injection can be largely eliminated when these biologics are administered with a hand-actuated aerosol generator. Numerous biologics are available and many of them can be made into a dry powder or liquid formulation, which then can be administered by using the newly invented hand-actuated aerosol generator. Without limitation, these common drugs are listed here as a part of the candidates for delivery using the newly invented hand-actuated aerosol generator: 90Y-lbritumomab tiuxetan, Abatacept, Abciximab, Adalimumab, ado-trastuzumab emtansine, Aflibercept, Agalsidase beta, Albiglutide, Aldesleukin, Alefacept, Alemtuzumab, Alemtuzumab, Alglucosidase alfa, Alteplase, Anakinra, asparaginase Erwinia chrysanthemi, Basiliximab, Becaplermin, Belatacept, Belimumab, Bevacizumab, Bortezomib, Brentuximab vedotin, Canakinumab, Capromab, Pendetide, Certolizumab pegol, Cetuximab, Collagenase, Collagenase Clostridium Histolyticum, Daclizumab, Darbepoetin alfa, Dasatinib, Denileukin diftitox, Denosumab, Domase alfa, Drotrecogin alfa, Dulaglutide, Ecallantide, Eculizumab, Efalizumab, Elosulfase alfa, Epoetin alfa, Eriotinib, Etanercept, Everolimus, Fanolesomab, Filgrastim, Galsutfase, Gefitinib, Gemtuzumab, Glucarpidase, Golimumab, Ibritumomab tiuxetan, Idursulfase, Imatinib, Infliximab, Interferon alfa-2a, Interferon alfa-2b, Interferon alfa-2b, Interferon alfacon-1, Interferon alfa-n3, Interferon alpha, Interferon beta-1a, Interferon Beta-1b, Interferon gamma-1b, Interleukin-2, Ipilimumab, Lapatinib, Laronidase, Lenalidomide, Methoxypolyethylene glycol epoetin beta, Metreleptin, Muromonab-CD3, Natalizumab, Nofetumomab, Obinutuzumab, Ocriplasmin, Ofatumumab, Omalizumab, Oprelvekin, Palifermin, Palivizumab, Palivizumab, Panitumumab, Pegaspargase, Pegfilgrastim, Peginterferon alfa-2a, Peginterferon alfa-2b, Peginterferon beta-1a, Pegloticase, Pembrolizumab, Pertuzumab, Ramucirumab, Ranibizumab, Rasburicase, Raxibacumab, Reteplase, Rilonacept, Rituximab, Sargramostim, Sittuximab, Sorafenib, Sunitinib, tbo-filgrastim, Tenecteplase, Thalidomide, Tocilizumab, Tositumomab, Trastuzumab, Ustekinumab, Vedolizumab, Vemurafenib, ziv-aflibercept.

If the current biologics are made in the injection formulation, the dosage range of each of these biologics in a formulation for using a hand-actuated aerosol generator is between 5.0 times higher and 5.0 times lower than its actual amount in the currently-known injection formulation. Preferably, the same amount of the actual biologic substance is needed in a dose-maker of a hand-actuated aerosol generator as in its corresponding injection formulation.

If the current biologics are made in the inhalation formulation, the dosage range of each of these biologics in the dose-maker of a hand-actuated aerosol generator is between 2.0 times higher and 2.0 times lower than its actual amount in the currently-known inhalation formulation. Preferably, the same amount of the actual biologic substance is needed in a dose-maker of a hand-actuated aerosol generator as in its corresponding known inhalation formulation.

Glutathione is an essential anti-oxidant found predominately in the brain and liver. When given intravenously it improves liver and brain function. If given via pulmonary route, glutathione will be absorbed into blood quickly, the same effect as IV administration. Pulmonary glutathione administration can be used for memory enhancement, dementia, multiple sclerosis, Parkinson's disease, neuropathy and liver disease. Glutathione has been shown to help slow the process of nerve cell degeneration. Levels of glutathione, a naturally occurring brain-protecting antioxidant, are significantly decreased in Parkinson's patients, with the deficiency occurring in the portion of the brain where dopamine-generating neurons are concentrated. For many Parkinson's patients, glutathione replenishment has proven to be an effective therapy for halting or even potentially reversing disability. In patients who respond to IV therapy, the effects can be almost immediate and dramatic. Within an hour of treatment, patients often experience reduced rigidity, fewer tremors, and improved walking ability. Pulmonary glutathione therapy can achieve the same effect.

Other anti-oxidant therapies delivered via pulmonary route. Deterioration of sight is one of the most common health problems of aging. Around age sixty, the likelihood of serious ocular disorders—macular degeneration, cataracts, and diabetic retinopathy—accelerates. Fortunately, these vision problems can be slowed and even reversed in many cases. Because free radical damage is a significant contributor to these problems, antioxidants have a strong protective effect. Over the past years, several studies have shown that the risk of macular degeneration, retinopathy, and cataracts can be greatly reduced using antioxidant supplementation. Pulmonary administration of high doses of antioxidants, fatty acids, carotenoids, amino acids, and minerals (sometimes in conjunction with chelation therapy) can dramatically enhance nutrient delivery and arrest degeneration of vision.

A new group of pulmonary therapies are to replace IV (intravenous injection or infusion) or IM (intramuscular injection) nutritional therapies. It is well known that vitamin B12 deficiency can be treated by either IM or IV administration of B12. Vitamin C and many other vitamins and minerals previously administered via IM or IV can be replaced, at least in part, by pulmonary delivery using the current invention, or newly termed as pulmonary nutritional therapy (PNT).

PNT can be used to increase energy, repair enzyme systems and promote optimum nervous system function. These nutrients include but not limited to any vitamins, such as vitamin C, B vitamins, and magnesium, as well as other minerals and trace minerals.

PNT can be beneficial for asthma, migraines, hepatitis, fibromyalgia, muscle spasm, chronic fatigue, upper respiratory infection, Parkinson's disease, malnutrition, anti-aging, allergic rhinitis, chronic sinusitis, acute viral illness, Herpes outbreaks, acute strain or injury, coronary artery disease (hypertension), detoxification (including heavy metals), depression, high cholesterol, high blood pressure, diabetes, obesity, etc.

PNT can be used periodically in healthy people to enhance overall well-being. As oral administration of certain nutrients can be too slow for a demanded quick recovery for sport athlete as an example. PNT, on the other hand, is the more suitable choice and capable of meeting the speedy and efficacy needs in providing nutrients to the body. Additionally, orally administered vitamins and nutrients are converted or broken down in the stomach and liver during the absorption process at high doses often cause gastrointestinal symptoms and diarrhea adverse side effects. In comparison, when using PNT, nutrients with a small but effective dose directly enters into the bloodstream in an unaltered, safe form and very well tolerated, with minimal or no side effects.

Pulmonary magnesium therapy for heart attack, heart failure and hypertension. If anyone needs to be rushed to the hospital with a heart attack, pulmonary inhalation of magnesium could save the life. In a 1995 study, researchers found that the in-hospital death rate of those receiving IV magnesium was one-fourth that of those who received standard treatment alone. In 2003, a follow-up study of these same type of the patients revealed an enduring effect of magnesium treatment. Nearly twice as many patients in the standard treatment group had died compared to those who received magnesium, and there were considerably more cases of heart failure and impaired heart function in the placebo group. Pulmonary administration of magnesium can be as protective as IV administration. Pulmonary administration of magnesium can be easily done at home, much earlier than IV administration which needs to be dome in the hospital or clinic by a medical professional. For this life-saving purpose, every minute counts. A simple way of administration of magnesium has obvious advantages. In addition to increasing survival after heart attack, IV magnesium can also smooth out arrhythmias and improve outcomes in patients undergoing angioplasty with stent placement. Pulmonary administration of magnesium is also beneficial for acute asthma attacks, often working to relax airway spasms when drugs do not. Pulmonary administration of magnesium is crucial for diabetics because it improves insulin sensitivity, helps blood sugar control, and reduces risk of retinopathy. Pulmonary administration of magnesium, therefore, can be used to meet these needs. Magnesium can also reduce the frequency and severity of migraine headaches, help prevent kidney stones, boost immune function, and protect DNA from carcinogens. Magnesium can even help sleep. It not only relaxes the muscles, but it also increases the length of restorative slow-wave sleep.

Pulmonary diabetic nutritional therapy can be used to improve glucose metabolism, improve neuropathy, and vascular disease associated with diabetes. Many nutrients, such as alpha lipoic acid, folic acid, B12, or thiamine, can be self-administered via pulmonary delivery using the current invention.

Everyone's immune system is on constant guard to protect against viruses, bacteria, and other pathogens. This system keeps the body free of not only serious infections but also cancer. Lupus and rheumatoid, in which the immune system attacks healthy cells, need a well-managed nutritional therapy. A number of nutrients play well-defined roles in the immune response, such as antioxidants, vitamins, and minerals. For patients suffering from immune disorders—or anyone who just needs an immune boost, pulmonary delivery of nutrients offers great help, much easier than IV administration. Our unique pulmonary cocktails can be made to contain high doses of B-vitamins along with vitamins C, E, beta-carotene, selenium, zinc, amino acid, nucleosides, nucleotides, plus glutathione, all of which are vital for a healthy immune system.

High dose vitamin C is used for cancer co-management therapies, fatigue, fibromyalgia, immune dysfunction and pre- and post-mercury amalgam removals. Intravenous or pulmonary vitamin C is especially effective in fighting viruses and cancer cells, while being very safe for all normal cells. It is because of this characteristic that IV and/or pulmonary delivery of vitamin C is particularly useful in knocking out the viruses of the common cold, flu, and sore throat. Patients often start feeling better within hours of administration of vitamin C.

Myer's Cocktail—a special therapy developed by Dr. John Meyer, a physician at Johns Hopkins University, contains multiple vitamins and minerals for IV administration. The cocktail is indicated for improving chronic fatigue, fibromyalgia, depression, muscle spasm, asthma, hives, congestive heart failure, angina (chest pain), infections, and senile dementia. Myer's Cocktail can be easily administered via the pulmonary route by using the current invention.

Phospholipids are primary constituents of the membranes that surround each of our cells, protecting and controlling what enters them. Among the most essential phospholipids is phosphatidylcholine (PC), which is the backbone of Plaquex. As one ages, his/her production of PC and other phospholipids falters, which has adverse effects throughout the body. A modified Plaquex therapy by administration of nutrition via the pulmonary route, not IV, can replenish the supply of these crucial building blocks, and can have the ability to decrease plaque deposits in arteries, improve exercise tolerance, reduce angina attacks, lower cholesterol and triglyceride levels, boost circulation, increase male potency, and improve kidney function. Anyone with heart disease, diabetes, arterial blockages, sexual dysfunction, high cholesterol, or lipid problems can greatly benefit from the modified Plaquex therapy via self-administration of nutrients via pulmonary delivery by using the current invention.

IV nutritional therapy has been used for PMS, menopause helpful for mood swings, irritability, insomnia, depression, cramps, etc. IV nutrients including B complex, pyridoxine, and magnesium. These nutrients can be administered easily via the pulmonary route by using the current invention.

Alpha-lipoic acid is an antioxidant that is made by the body and is found in every cell, where it helps turn glucose into energy. Antioxidants are substances that attack free radicals, waste products created when the body turns food into energy. Free radicals cause harmful chemical reactions that can damage cells in the body, making it harder for the body to fight off infections. They also damage organs and tissues. Unlike other antioxidants, which work only in water (such as vitamin C) or fatty tissues (such as vitamin E), alpha-lipoic acid is both fat- and water-soluble. That means it can work throughout the body. In addition, antioxidants are depleted as they attack free radicals, but evidence suggests alpha-lipoic acid may help regenerate these other antioxidants and make them active again. Alpha-lipoic acid has been administered by IV along with silymarin to treat people who have eaten the poisonous mushroom Amanita, which causes liver damage. Using the current invention, alpha-lipoic acid can be easily administered and enter into blood circulation via the pulmonary route.

The new trend towards the safer and more effective therapy is to use the combinational active ingredients. Aerosol inhalation by using a hand-actuated aerosol generator offers a broad range of new applications. Although many existing DPIs have been used for many years to deliver one or two chemical drugs, the new hand-actuated aerosol generator can replace the DPI for a much better performance. The hand-actuated aerosol generator can also be used to deliver a mix of multiple agents to improve the therapeutic effect. In addition, using a hand-actuated aerosol generator can deliver the new combination of the chemical drug and the extract from one or more herbal medicines. A number of ingredients can be combined, micronized, formulated, mixed, homogenized, packed and then loaded into a hand-actuated aerosol generator for inhalation. Some of these ingredients are bronchodilator, antimicrobial agent, anti-inflammatory agent, anti-allergic agent, antihistamine agent, immunomodulation agent, tissue healing agents, and any of the needed nutrients. Many fine extracts of the formulated TCM herbs can be used in combination. Using an appropriate process to produce the right particle sizes such as 2-10 micron, these therapeutic agents can be incorporated into the dry powder and delivered into the lower respiratory tract with a hand-actuated aerosol generator. Different formulations with the correctly selected active ingredients can be made to meet the different needs for different patients with different diseases, such as diabetes.

In one aspect, the present invention provides a number of new therapies offered through the combinational use of a medical device—a hand-actuated aerosol generator and correctly selected active therapeutic ingredients. It is known that developing a new formulation delivered with the metered dose inhaler (MDI) has a number of technical challenges particularly due to the fact that the propellant may not be safe, and preservative agents are needed to maintain a required long shelf-life of the liquid drug. Formulation of the powder delivered with a DPI in general is easier than that delivered with a MDI. Due to the reduced need for using other components, the formulated drug in the dry powder form can be much purer than the drug in the liquid form. When it is appropriate, the pure active ingredient(s) alone can be delivered with a hand-actuated aerosol generator. In practice, any drugs delivered using a MDI or DPI now can be delivered by using a hand-actuated aerosol generator since the need for a strong inhalation capacity for using a breath-actuated DPI no longer exists with the hand-actuated aerosol generator.

In another aspect, the present invention is for a patient who is suffering from a hard to treat asthma that is not responsive to the common therapy or the standard of care treatment. The new combination of several protective active ingredients will improve the response rate. Administering to the patient an effective amount of the combinational ingredients directly to the lungs through a hand-actuated aerosol generator can potentially quickly result in the symptom relief.

Preferably, the active ingredient(s), such as epinephrine, is administered by using a hand-actuated aerosol generator for the immediate relief of asthma symptoms or acute allergic reaction, such as anaphylaxis. The patient is not required to have a strong inhalation capacity in using a hand-actuated aerosol generator, which is critically important for saving the patient when a severe allergic reaction occurs.

In yet another aspect, the present invention provides a method for treating respiratory diseases by administering an effective amount of the active ingredient in a fine powder form or a liquid form to a patient in need of such a treatment where the respiratory diseases or one or more manifestations of the respiratory diseases are non-responsive or substantially non-responsive to treatment with the current standard of care. Additionally, the present invention provides a method of treating a severe and long-lasting episode of asthma by administering an effective amount of the active ingredient to a patient in need of such a treatment, where the severe and long-lasting episode of asthma or one or more manifestations of it are non-responsive or substantially non-responsive to treatment with the standard of care. Within the present invention, in one embodiment, the active ingredient administered is an herbal formula alone or in combination with a chemical drug, with or without using a pharmaceutically acceptable carrier. For example and without limitation, patients suffering from respiratory diseases (for example, those admitted to an emergency room because of an acute exacerbation of asthma), who are not responsive to the standard of care, can be treated with a combinational herbal formula and modern medicine (in addition to having been treated with the standard of care) and their treatment outcomes could be more favorable.

The particle size of any dry powder has a significant impact on the therapeutic result due to its distribution in the respiratory tract. For delivery using a hand-actuated aerosol generator, the particle size of the active ingredient(s) should be within 2-5 microns. It is well known in the pharmaceutical industry that the powdered medicament particles suitable for delivery to the bronchial or alveolar region of the lungs have an aerodynamic diameter of less than 10 micrometers, preferably within the range of 2 to 5 micrometers. Particles of powdered medicament and/or excipient may be produced by conventional techniques, for example by spray drying micronization, milling or sieving.

Other sized particles may be used if delivery to other parts of the respiratory tract is desired, such as the nasal cavity, mouth or throat. However, for treating any disease or disorder systemically, better results will generally result from pulmonary delivery so the therapeutics can enter into the blood circulation more quickly. The medicament may be delivered as a pure drug, or more appropriately, it is preferred that medicaments are delivered together with excipients (carriers) which are suitable for inhalation. Suitable excipients include organic excipients such as polysaccharides (e.g. starch, cellulose and the like), lactose, glucose, mannitol, amino acids, and mattodextrins, and inorganic excipients such as calcium carbonate or sodium chloride. Lactose is a commonly used and preferred excipient. Additionally, medicament and/or excipient powders may be engineered with particular densities, size ranges, or characteristics. Particles may comprise active agents, surfactants, wall forming materials, or other components considered desirable by those of ordinary skill in the art.

Patients who have normal lungs will experience an ease in breathing with a powder formulation for a quick therapeutic effect, even when using a breath-actuated DPI. After the accuracy of drug dose delivery is ensured by using the new hand-actuated aerosol generator, more patients will prefer this better and more user-friendly device in order to receive the medicine they need into their lungs. It is well known that the inhaled medicine works faster than the same medicine in a pill. In addition, the patients will likely need a smaller amount of the medicine than they would orally, hence, side effects caused by excessive drug substances can be reduced or even eliminated due to the smaller exposure to the given drug. Thus, regarding potential side effects, hand-actuated aerosol generator delivery is expected to be advantageous than swallowing the pill.

The newly invented hand-actuated aerosol generator is a medical device delivering significant improvements in aerosol delivery including better standardization of delivery force, device function and patient use, greater reliability, more accurate dosing, and reduction of drug loss and potential adverse effects.

It is easy for the majority of adults and children 4 years and older either alone or under the supervision of an adult to use a hand-actuated aerosol generator.

Multiple dry-powder or liquid drugs can be included in a cocktail and can be included in one single capsule or a vial by using the current invention. This will create a huge amount of new therapies, such as for treating hypertension, diabetes, obesity, heart diseases, infections, etc. The cocktail treatment will become very popular, gradually, as the synergy among several drugs at the lower dose of each, and the mix can have the therapeutic effect while it is only in one-time administration with one drug preparation. Below is the method and procedure of using a natural therapeutic kit containing a hand-actuated aerosol generator and an inhalable therapeutic natural powder, or using a modern therapeutic kit containing a hand-actuated aerosol generator and an inhalable pharmaceutically active powder, or using a therapeutic biologic kit containing a hand-actuated aerosol generator and an inhalable pharmaceutically active biologic powder, or using a therapeutic herb kit containing a hand-actuated aerosol generator and an inhalable herb powder, or using a combinational therapeutic kit containing a hand-actuated aerosol generator and an inhalable powder of the combinational mix:

Step 1. Load or make sure the inhalable medicament is loaded in the container of the hand-actuated aerosol generator;

Step 2. Take an exhalation;

Step 3. Place the mouthpiece inside the mouth;

Step 4. Use hand to apply the positive pressure, or turn on the supplier of the positive pressure, to trigger the aerosol release of said medicament;

Step 5. Start a gentle inhalation at the same time of applying positive pressure to trigger the aerosol release of said medicament;

Step 6. If needed, after a gentle exhalation, repeat one or more times of steps 4 and 5;

Step 7. Rinse mouth and clean the mouthpiece for future use.

### EXAMPLES

The following examples are provided to illustrate certain aspects of embodiments of the present invention, and to aid those of ordinary skill in the art in practicing embodiments the invention. These examples are not intended to limit the scope of the invention as these and other equivalent embodiments will be apparent in view of the present disclosure, figures, and accompanying claims.

### Example 1: A Natural Herb Kit for Treating Rheumatoid Arthritis

A preferred embodiment of the kit contains a hand-actuated aerosol generator and 1000 mg dry powder extracted from Tripterygium wilfordii, a traditional Chinese herbal medicine. The herb, Tripterygium wilfordii, is completely dried, broken down to very tiny pieces, placed into water, heated to the boiling point for 20 minutes and then kept overnight. The solid waste is removed and the extract is processed by spray drying to obtain the fine powder in the size of 2 to 10 micron. The micronized powder of 1000 mg is then mixed with 4000 mg inhalable lactose and homogenized. The mix in the total weight of 5000 mg is packed into the hand-actuated aerosol generator kit. This kit can be used by a patient with rheumatoid arthritis daily without painful daily injection.

### Example 2: A Natural Therapeutic Kit for Treating Asthma or Improve Immunity

A preferred embodiment of the kit contains a hand-actuated aerosol generator and an inhalable therapeutic natural powder made from mixing and homogenizing the extract in the powder form of six herbs for making the commonly used Chinese medicine Lui-Wei-Di-Huang Wan (LWDHW). One thousand milligrams of each fine powder of the extract of Cortex Moutan Radicis (Paeonia Suffruticosa) (Mu Dan Pi), Fructus Comi Officinalis (Shan Zhu Yu), Radix Rehmanniae Preparata (Shu Di Huang), Rhizoma Alismatis Orientalis (Ze Xie), Rhizoma Dioscoreae Oppositae (Shan Yao), and Sderotium Poriae Cocos (Fu Ling) are mixed and homogenized, milled into a very fine powder. The total 6000 mg mix is packed into a hand-actuated aerosol generator kit. This kit can be used regularly by a patient with asthma or by someone who needs to improve immunity.

### Example 3: A Therapeutic Powder Formulation for Diabetes

A preferred embodiment of the therapeutic powder formulation for diabetes is made by combining the fine powder of the extract (500 mg each) from adenophora, anemarrhena, asparagus root, dendrobium, glehnia, gypsum, lycium bark, ophiopogon, pueraria, raw rhmannia, scrophularia, trichosanthes root, and yu-chu. The mix is micronized and homogenized, then packed into a hand-actuated aerosol generator for oral inhalation on a daily basis.

### Example 4: A Therapeutic Powder Formulation for Digestive Disorder

A preferred embodiment of the therapeutic powder formulation for digestive disorder is made by combining the fine powder of the extract (1000 mg each) from astragalus, atractylodes, dioscorea, ginseng, polygonatum, and pseudostellaria. The mix is homogenized, then packed into a hand-actuated aerosol generator kit for oral inhalation on an as-needed basis.

### Example 5: A Therapeutic Powder Formulation for Cleansing Toxicant in the Liver and Kidneys

A preferred embodiment of the therapeutic powder formulation for cleansing toxicant in the liver and kidneys is made by combining the fine powder of the extract (1000 mg each) from aconite, alisma, cinnamon, comus, epimedium, ho-shou-wu, and lycium fruit. The mix is homogenized, then packed into a hand-actuated aerosol generator kit for oral inhalation during the therapeutic period.

### Example 6: A Therapeutic Powder Formulation for Treating Abnormal Blood Triglyceride

A preferred embodiment of the therapeutic powder formulation for treating abnormal blood triglyceride is made by combining and mixing the fine powder of the extract (1000 mg each) from astragalus, polygonatum, pseudostellaria, rehmannia, and trichosanthes root. The mix is homogenized, then packed into a hand-actuated aerosol generator kit for oral inhalation during the therapeutic period.

### Example 7: A Therapeutic Powder Formulation for Improving Peripheral Blood Circulation

A preferred embodiment of the therapeutic powder formulation for improving peripheral blood circulation is made by combining and mixing the fine powder of the extract (500 mg each) from anemarrhena, astragalus, atractylodes (cangzhu), carthamus, codonopsis, gypsum, persica, rehmannia, salvia, and tang-kuei. The mix is homogenized, then packed into a hand-actuated aerosol generator kit for oral inhalation during the therapeutic period.

### Example 8: A Therapeutic Kit for Relieving Respiratory Symptoms

A preferred embodiment of the therapeutic kit for relieving respiratory symptoms is made by mixing a dry powder of epinephrine and inhalable lactose in a ratio of 0.1 and 99.9. The 5000 mg mix is homogenized and micronized, and then packed into a hand-actuated aerosol generator kit for pulmonary administration. This therapeutic kit can be used by a patient with asthma or with a food allergy on an as-needed basis.

### Example 9: A Combinational Therapeutic Kit for Relieving Respiratory Symptoms

A preferred embodiment of the therapeutic kit for relieving respiratory symptoms is made by first mixing a dry powder of epinephrine and inhalable lactose in a ratio of 0.2 and 99.8. Then, the mix is further mixed with an extract of licorice in a very fine powder form in a 1:1 ratio. The total mix weight will be 5000 mg. The 5000 mg mix is homogenized and micronized, and then packed into a hand-actuated aerosol generator kit for pulmonary administration. This therapeutic kit can be used by a patient with asthma or with a food allergy on an as-needed basis.

### Example 10: A Combinational Therapeutic Powder Formulation for Balancing Blood Sugar Concentration

A combinational therapeutic powder formulation for balancing blood sugar concentration is made by combining and mixing the fine powder of the extract (500 mg each) from alisma, atractylodes, coptis, ginseng, ho-shou-wu, lonicera, lycium bark, phellodendron, polygonatum, rehmannia, scrophularia, and yu-chu, alone with epinephrine (0.25 mg). The mix is homogenized and micronized, and then packed into a hand-actuated aerosol generator kit for oral inhalation. This therapeutic kit can be used by a patient with hyperlipidemia on a daily regular basis.

### Example 11: A Therapeutic Kit for Type 1 Diabetes

A preferred embodiment of the therapeutic kit for type 1 diabetes contains a hand-actuated aerosol generator and an inhalable insulin in the fine powder form. Insulin is mixed with lactose as a carrier. Insulin dose will be in the amount of 8 units per inhalation. This is for oral inhalation using a hand-actuated aerosol generator kit at each meal to balance blood glucose level. The 8 units inhaled insulin is to replace 8 units of subcutaneous injection of insulin at the beginning of the mealtime.

### Example 12: A Therapeutic Kit for Type 2 Diabetes

A preferred embodiment of the therapeutic kit for type 2 diabetes contains a hand-actuated aerosol generator and an inhalable insulin in the fine powder form. Insulin is mixed with lactose as a carrier. Insulin dose will be in the amount of 4 units per inhalation. This is for oral inhalation using a hand-actuated aerosol generator at each meal to balance blood glucose level. The 4 units inhaled insulin is to replace 4 units of subcutaneous injection of insulin at the beginning of the mealtime.

### Example 13: A Therapeutic Kit for Influenza and Respiratory Tract Infection

A preferred embodiment of the therapeutic kit for influenza and respiratory tract infection contains a hand-actuated aerosol generator and an inhalable mixture of Shuang Huang Lian, a formula with three traditional Chinese medicines. This formula comprises three herbs: lonicera, scute and forsythia. This TCM formula is traditionally known as an antiviral, and is most commonly used for the treatment of respiratory infections, including upper and lower respiratory tract infections and acute bronchiolitis. The mix of the three extracts (each 2000 mg) in the very fine powder is homogenized and packed in hand-actuated aerosol generator kit for oral inhalation. This therapeutic kit can be used during any respiratory tract infection, including influenza or pneumonia.

### Example 14: A Therapeutic Kit for Relieving Respiratory Symptoms (Asthma or COPD)

A preferred embodiment of the therapeutic kit for relieving respiratory symptoms (asthma or COPD) is made by packing a hand-actuated aerosol generator and the mix of albuterol and inhalable lactose. Albuterol is mixed with the fine powder of inhalable lactose with or without licorice extract. The mix is homogenized and packed in capsules for pulmonary administration using a hand-actuated aerosol generator. Each inhalation will deliver 0.20 mg albuterol. This therapeutic kit can be used whenever needed by the patient with asthma or COPD.

### Example 15: A Risk-Reduction Kit for Preventing Symptoms of Bronchospasm Prophylaxis

A preferred embodiment of the risk-reduction kit for preventing symptoms of bronchospasm prophylaxis contains a hand-actuated aerosol generator and the mix of epinephrine and inhalable lactose. The mix is homogenized and packed in capsules for pulmonary administration using a hand-actuated aerosol generator. Each inhalation will deliver 0.20 mg epinephrine. This therapeutic kit is to replace epinephrine injection for relieving symptoms of bronchospasm prophylaxis. The user should use the kit 15 minutes before exercise.

### Example 16: Using a Therapeutic Kit for Relieving Respiratory Symptoms of Asthma

The volunteers with initials of JL and SM used the therapeutic kit containing a hand-actuated aerosol generator and the mix of epinephrine and inhalable lactose. Each inhalation delivers 0.11 mg of epinephrine, and two inhalations deliver 0.22 mg of epinephrine. About 1 to 5 minutes after oral inhalation of epinephrine, they noticed the physiological changes in their lungs.

### Example 17: Pulmonary Delivery of Nutrients

The volunteer with initials of JL used the therapeutic kit containing a cocktail of nutrients delivered by using a hand-actuated aerosol generator. Vitamin C, B1, B2, B6, biotin were included. Each inhalation delivers 50% of recommended amount of the daily requirement of the nutrient from the diet. It was well tolerated and no any noticeable side effects were experienced.

### Example 18: Using a Therapeutic Kit for Relieving Respiratory Symptoms of Asthma

The volunteer with initials of JL used the therapeutic kit containing a liquid made with sea salt, sodium bicarbonate, citric acid, sodium citrate, and vitamin C, delivered by using a hand-actuated aerosol generator. Each inhalation delivers 60 microliters of the liquid. It was easy to do and it was well tolerated and no any noticeable side effects were experienced.

It is understood that numerous workable combinations of features and elements of the hand-actuated aerosol generator, treatment mixes and therapeutic kits can be implemented according to the present invention. Practical implementations of the invention are numerous and not limited to the detailed embodiments and descriptions provided here, which are for the purpose of clarity and to enable the making of numerous new therapeutic kits and other products by practitioners skilled in the art, according to the invention.

1. A hand-actuated aerosol generator, comprising:
a. An adjustable two-compartment medicament container that holds a medicament in a liquid or a fine dry powder form, and connects to a fluid passage;
b. a cap of said container with a jet-sprayer capacity;
c. a within-space or dose-maker configured to house an adjustable but defined amount of medicament, wherein the within-space or dose-maker is filled by a turning the spiral rod fixed inside of said medicament container,
d. a one-way air inlet channel through configured such that pressured air can enter the narrow space to form jet-blow pressure, and a jet-sprayer head coupled to the air inlet channel;
e. a mouthpiece through which a medicament released from said jet-sprayer can be inhaled into the respiratory tract by a patient without leakage;
f. an optional spacer that increases inhalation efficiency.

2. The hand-actuated aerosol generator of claim 1, wherein the medicament container comprises an interior having a volumetric size ranging from about 0.5 ml to about 100 mi.

3. The hand-actuated aerosol generator of claim 1, wherein the volume of the within-space or dose-maker ranges from about 0.001 ml to about 5.0 mi.

4. The hand-actuated aerosol generator of claim 1, wherein said positive pressure supplier is (1) an air pump which is a hand-held squeezable bulb with an one-way valve which permits the air flow into said container only, not suck the medicament out of the chamber; and/or the second one-way valve in the bulb which permits air only enter into the squeezable bulb to generate positive pressure repeatedly; or, (2) an air pump that is powered with a battery or plug-in electricity with an on/off switch; or (3) a pressured-air filled container from which the positive pressure can be released upon turning on the switch of said pressured air to force said liquid or powder flow into said jet-sprayer to transform as aerosol for oral inhalation via said mouthpiece by a patient.

5. The hand-actuated aerosol generator of claim 1, said air-supplier for supplying positive pressure to a hand-actuated aerosol generator comprising: (1) a power supplier switched on/off with a hand, which is (a) an electronic power supplier; or (b) an elastic bulb onto which to apply pressing power by a human hand, or (c) a pressured-air container to release positive pressure upon turning on by a hand; (2) a mechanical apparatus including an air pressure generator and an air passage; (3) a valve mean; (4) an airflow adaptor switched on/off by a hand to connect to the airflow chamber of the positive pressure dry powder inhaler; (5) a switch mean; (6) a meter or apparatus to control, measure and indicate air pressure for accurately supplying positive pressure to the airflow chamber of the positive pressure dry powder inhaler.

6. A therapeutic kit, a drug-device combinational product, comprising (A) a hand-actuated aerosol generator for easy administration of the inhalable medicament packed in a single dose or in multiple doses, which comprises: a volume-adjustable drug container which also has an inner adjustable dose-maker to hold a dosed amount of medicament; a jet-sprayer head as a cap for said drug container which can generate aerosol in particle size from 1 to 10 micron; a positive air-pressure supplier, and a mouthpiece; (B) an inhalable pharmaceutically active ingredient for treating a human disease/disorder, such as epinephrine for treating asthma or COPD; and (C) an optional drug carrier, such as non-propellant saline or inhalable lactose.

7. The inhalable pharmaceutically active ingredient in the therapeutic kit of claim 6, wherein said ingredient, such as epinephrine, is in the fine powder form varying its particle size from 1 to 50 microns.

8. The inhalable pharmaceutically active ingredient in the therapeutic kit of claim 6, wherein said ingredient, such as epinephrine, is in a small amount about 0.01 to 550 mg, which is accounted for 0.1% to 100% of the total formulation weight.

9. The inhalable pharmaceutically active ingredient in the therapeutic kit of claim 6, wherein said ingredient is dissolved in a liquid without propellant, such as saline.

10. The inhalable pharmaceutically active ingredient in the therapeutic kit of claim 6, wherein said ingredient is a chemical compound, such as ibuprofen.

11. The inhalable pharmaceutically active ingredient in the therapeutic kit of claim 6, wherein said ingredient is a biological molecule, such as a respiratory syncytial virus antibody.

12. The inhalable pharmaceutically active ingredient in the therapeutic kit of claim 6, wherein said ingredient is a natural compound, such as Tanshinone I extracted from Danshen (Salvia miltiorrhiza Bunge).

13. The inhalable pharmaceutically active ingredient in the therapeutic kit of claim 6, wherein said ingredient is a pre-mix with at least two components, such as ibuprofen and Tanshinone I.

14. A method of treating a human disease by using a therapeutic kit which is a drug-device combinational product comprising (A) a hand-actuated aerosol generator for easy administration of the inhalable medicament packed in a single dose or in multiple doses, which comprises: a volume-adjustable drug container which also has an inner adjustable dose-maker to hold a dosed amount of medicament; a jet-sprayer head as a cap for said drug container which can generate aerosol in particle size from 1 to 10 micron; a positive air-pressure supplier, and a mouthpiece; (B) an inhalable pharmaceutically active ingredient for treating a human disease/disorder, such as epinephrine for treating asthma or COPD; and (C) an optional drug carrier, such as non-propellant saline or inhalable lactose.

15. The method of treating a human disease of the claim 14, wherein said disease is a cardiovascular disease, such as an ischemic stroke.

16. The method of treating a human disease of the claim 14, wherein said disease is a malignant disease, such as lung cancer.

17. The method of treating a human disease of the claim 14, wherein said disease is a mental disease, such as depression.

18. The method of treating a human disease of the claim 14, wherein said disease is a metabolic disease, such as diabetes mellitus.

19. The method of treating a human disease of the claim 14, wherein said disease is a respiratory tract disease, such as influenza.

20. The method of treating a human disease of the claim 14, wherein said disease is a degenerative disease, such as spinal hernia.
